(19) United States
(12) Patent Application Publication
(10) Pub. No.: US 2024/0358469 A1
(43) Pub. Date: Oct. 31, 2024

(54) METHOD AND SYSTEMS FOR CABLE ATTACHMENT DETECTION

(71) Applicant: St. Jude Medical, Cardiology Division, Inc., St. Paul, MN (US)

(72) Inventors: Peter C. Mooers, Brooklyn Park, MN (US); Darren C. Carter, Shoreview, MN (US)

(21) Appl. No.: 18/644,837

(22) Filed: Apr. 24, 2024

Related U.S. Application Data
(60) Provisional application No. 63/462,374, filed on Apr. 27, 2023.

(51) Int. Cl. A61B 90/00

(52) U.S. Cl. CPC A61B 90/08; A61B 2090/0808

(57) ABSTRACT

A method of detecting whether an external wire is connected to an input terminal of an electrical system includes monitoring a signal associated with the input terminal, converting the analog signal to a digital signal, and processing the digital signal through Fast Fourier Transform (FFT). The method further includes detecting whether an external cable is connected to the input terminal based on the spectral content derived from the FFT. The method can include generating a notification if an external wire is detected and implementing safety protocols that allow or prohibit delivery of electrical power to one or more external components, based on whether the external wire is detected. In an example, the electrical system is part of a medical system, and the external wire can be configured for attachment of a catheter.

### CROSS-REFERENCE TO RELATED APPLICATIONS

This application claims benefit of priority to U.S. Provisional Patent Application No. 63/462,374, filed on Apr. 27, 2023, the entire disclosure of which is hereby incorporated by reference.

### TECHNICAL FIELD

The present invention relates generally to electrical systems and methods and systems of detecting whether a wire or cable is attached to an input terminal of the electrical system.

### BACKGROUND

In medical applications, a catheter is typically connected to an external system such as an amplifier system or signal generator system. The external system may be utilized to analyze signals received from the catheter and/or to deliver signals to the catheter. In some cases, it can be important or beneficial to confirm whether the catheter is properly connected to the amplifier system. For example, it may be beneficial to know if the catheter is connected or not to the external system during initialization or start-up of a signal generator (such as a high-voltage irreversible electroporation generator). It would therefore be beneficial to provide a system/method of automatically detecting whether the catheter, or more generally the corresponding cable or wire, is connected to the external system.

### SUMMARY

According to one aspect, a method of detecting whether an external wire is connected to an input terminal on an external medical system includes monitoring a signal associated with the input terminal, the input terminal located on an exterior panel of the external medical system and configured for receiving an external wire, and analyzing spectral content of the monitored signal. The method can further include detecting whether the external wire is connected to the input terminal based on the spectral content of the monitored signal.

According to another aspect, a method of operating an electrical system configured to provide power to one or more external components includes monitoring a signal associated with an input terminal on an amplifier system of the electrical system, wherein the input terminal is configured for connection to an external wire, analyzing spectral content of the monitored signal, and determining whether the external wire is connected to the input terminal based on the spectral content of the monitored signal. The method can further include performing at least one of: generating a notification relaying whether the external wire is connected and implementing safety protocols, if the external wire is connected.

According to another aspect, a system configured to provide electrical power to one or more external components includes one or more input terminals formed on an exterior panel of the system, the one or more input terminals configured to receive an external wire, and a data collection system configured to monitor and collect analog signals associated with the one or more input terminals. The analog signals can undergo digital signal processing in the data collection system to provide spectral content for the one or more input terminals. The system can further comprise a cable detection module configured to detect whether the external wire is connected to the one or more input terminals based on the spectral content.

### BRIEF DESCRIPTION OF THE DRAWINGS

FIG. 1 is a diagrammatic depiction of a medical system including an external electric system configured to interact with a medical device and a display workstation according to some embodiments.

FIG. 2 is a diagrammatic depiction of the external electrical system in FIG. 1 having one or more input terminals configured to interface/connect with a medical device according to some embodiments.

FIG. 3 is a diagrammatic depiction illustrating in more detail the external electrical system of FIG. 2 according to some embodiments.

FIG. 4A is a frequency plot illustrating spectral content for a particular channel of an input terminal with an external wire connected.

FIG. 4B is a similar frequency plot for the same channel as FIG. 4A, without a wire connected.

FIG. 5A is a frequency plot illustrating spectral content for a channel without a wire connected but adjacent to the channel of FIGS. 4A and 4B with a wire connected to the channel of FIGS. 4A and 4B.

FIG. 5B is a frequency plot for the same channel as FIG. 5A showing spectral content when the wire is not connected to the channel of FIGS. 4A and 4B.

FIG. 6 is a flowchart illustrating steps utilized to detect whether a cable is attached to an input terminal on the external electrical system.

### DETAILED DESCRIPTION

According to some embodiments, the claimed invention facilitates determining whether an external cable or wire is attached to an input terminal of an external system based on frequency analysis (e.g., spectral content) of the signal received at the input terminal. The spectral content (e.g., based on frequency domain analysis of the signal received at the input terminal) increases in response to connection of a device or component to the input terminal. The increase in spectral content is due to the wires, extending along a length of the device, responding to external electromagnetic radiation. When the device is disconnected from the external system the spectral content decreases. As such, the change or difference in spectral content makes it feasible to determine whether a device is connected to one of the input terminals.

In some embodiments, the external system and internal components associated therewith are housed within a metal/conductive frame that acts as a Faraday cage and prevents external electromagnetic radiation from generating spectral content or noise within the external system. In some embodiments, the signal received at the input terminal is analyzed utilizing a Fast Fourier Transform (FFT) that converts a time-domain signal into a frequency domain signal. One of the benefits of the claimed invention is that FFT modules do not add significantly to the cost of the external system, and such modules are often already included in the external system; therefore, the claimed invention provides a relatively inexpensive means of cable detection.

The method and systems disclosed herein can be used in a variety of electrical systems to detect whether an external wire or cable is attached to the electrical system, regardless of whether detection can be visually confirmed. For example, in medical applications, it may be important to ensure that a catheter and any corresponding external cables or wires are not attached to the external system prior to performing a start-up or self-test of the system. In other examples, it may be beneficial to confirm that a catheter or other component IS connected to the external system. In another example, it may be helpful to a user to confirm whether a foot pedal configured to operate a medical system is properly connected to an input terminal of the external system, prior to the pedal being utilized by a technician/physician. It yet another example, the methods and systems can be used in a system that monitors bodily signals. In some embodiments, the method and systems can be used to detect whether a cable that looks connected is in fact properly connected such that it is functioning properly or effectively. In some embodiments in which a cable contains multiple wires that connect to a single input terminal, the methods and systems can be used to determine if all of the wires are connected and working properly and differentiate which of the wires may not be functioning properly.

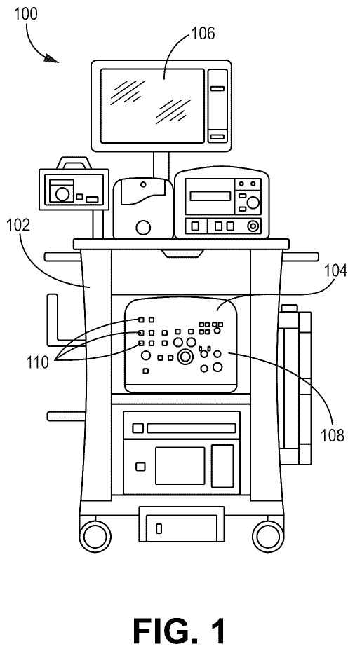

FIG. 1 is a diagrammatic depiction of a medical system 100 including a stand 102 for housing components of the system 100, an electrical system 104, and a display 106. The system 100 includes additional components shown in FIG. 1 that are not focused on herein. The system 100 can be configured for mapping, recording, localization, and ablation, such as radiofrequency (RF) and irreversible electroporation (IRE) ablation.

In some embodiments, the system 100 can be configured for providing one or more of mapping/localization/ablation therapy to the heart of a patient. In some embodiments, the electrical system 104 can be an amplifier. A catheter (not shown) can be connected to the electrical system/amplifier 104 via a wire or cable, and the catheter can deliver high voltage to the heart to provide IRE therapy to cardiac tissue. The voltage can be generated by a signal generator housed within the electrical system 104. It may be beneficial in some instances to initiate or perform start-up tests on the signal generator prior to providing ablation therapy. The method and systems disclosed herein facilitate catheter detection based on frequency analysis.

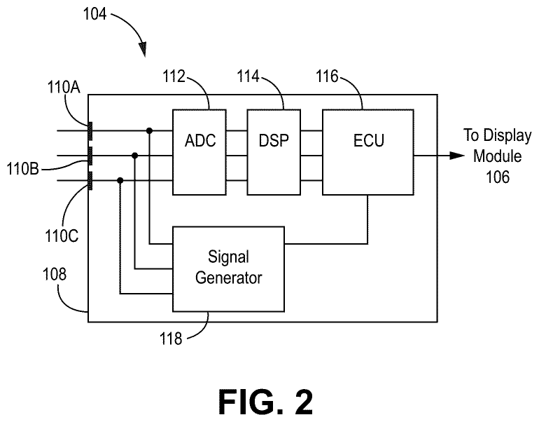

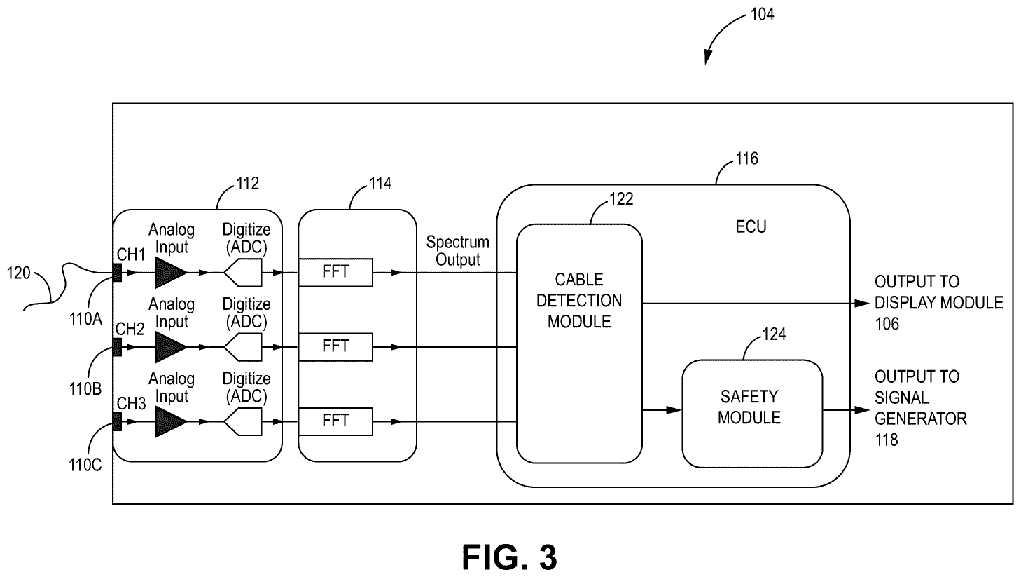

A front panel 108 of the electrical system 104 can include one or more input terminals or ports 110 configured to receive an external cable or wire that can be associated with a catheter. Additional components of the electrical system are described further below in reference to FIGS. 2 and 3.

FIG. 2 is a simplified schematic of the components inside the electrical system 104, which can include an analog to digital converter (ADC) 112, a digital signal processor (DSP) 114, an electronic control unit (ECU) 116 and a signal generator 118. In some embodiments, the ECU 116 includes a processor (e.g., application-specific integrated circuit (ASIC), microcontroller, graphical processor, etc.) and memory for storing instructions utilized by the processor to implement the functions and/or modules described herein. FIG. 2 shows three input terminals 110-specifically, terminals 110A, 110B, 110C, such that when a component is plugged into one of input terminals 110, the component can be electrically connected to the ADC 112 and the signal generator 118. Three input terminals 110 are shown in FIG. 2 but it is recognized that in other embodiments the electrical system 104 can include more or less input terminals. The ADC 112 and DSP 114 have both data collection and data processing capabilities and can collectively be referred to as a data collection system. The ECU 116 can include cable detection capabilities as discussed further below in reference to FIG. 3.

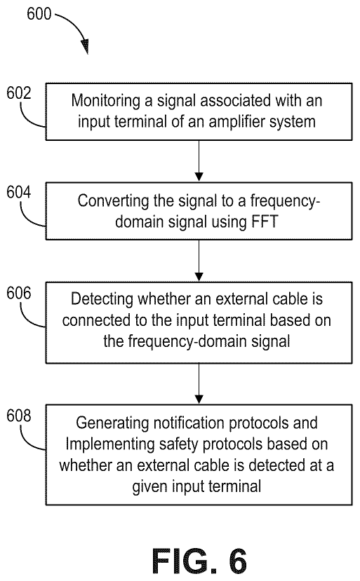

The exterior of the electrical system 104 can be constructed of metal such that the electrical system 104 acts as a Faraday cage, thus shielding the components inside the system 104 (e.g., ADC 112, DSP 114, ECU 116, and signal generator 118). The electrical system 104 can be connected to and communicate with the display 106 (see FIG. 1) such that the display 106 can provide outputs to a user of the system 100. This is described further below in reference to FIGS. 3 and 6.

FIG. 3 is an alternative schematic to FIG. 2 of the components inside the electrical system 104, providing additional detail relative to FIG. 2. In FIG. 3, a wire 120 is shown attached to one of the input terminals 110, specifically to input terminal 110A. In some embodiments, each of the input terminals 110A, 110B and 110C can correspond to a particular channel, as described further below. In some embodiments, the wire 120 can correspond to and be attached to a device, such as a catheter. In some embodiments, the catheter is attached to the front panel 108 via a single wire like the wire 120 in FIG. 3. In other embodiments, the catheter can be attached to the front panel via a cable, the cable configured to house multiple wires that correspond to multiple receptacles formed in the input terminal.

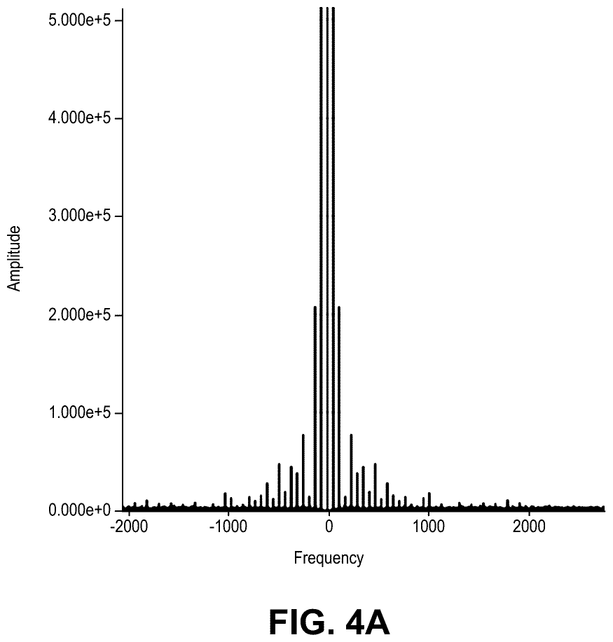

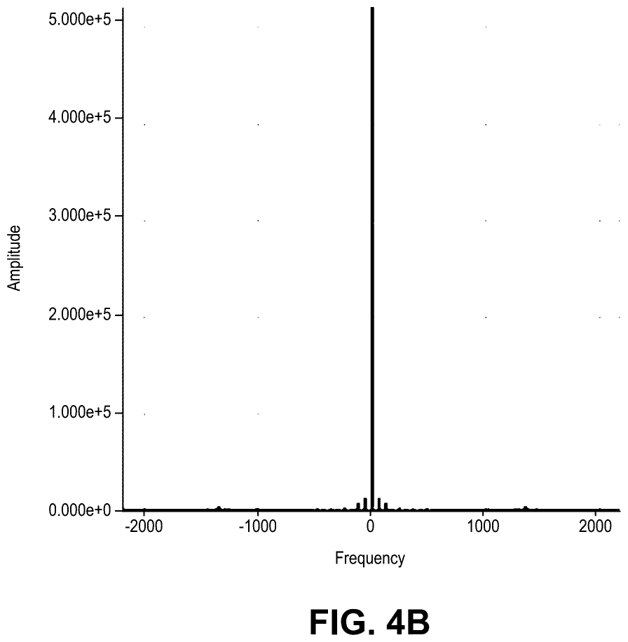

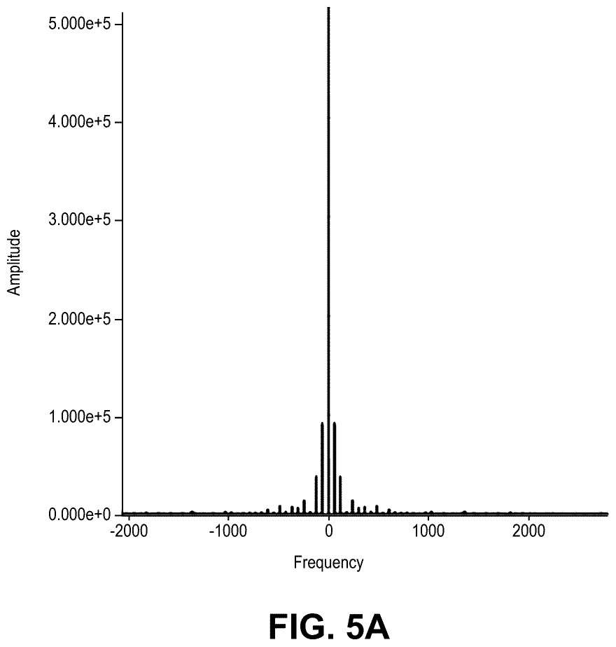

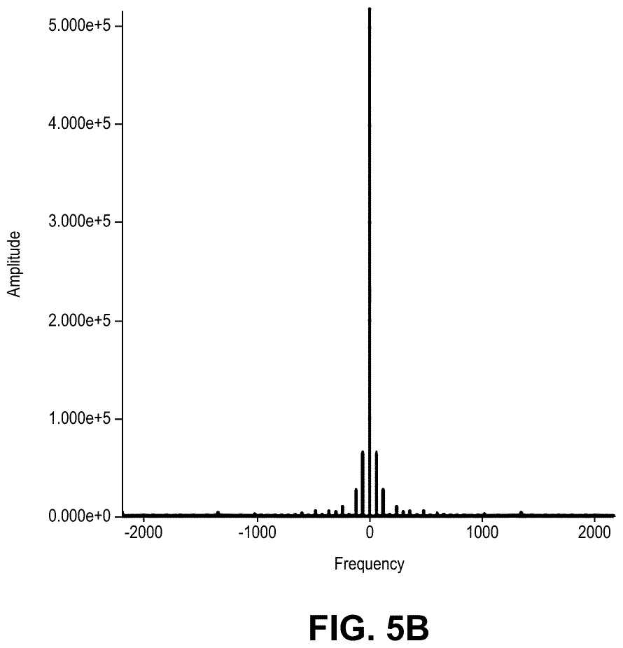

In the ADC 112, an analog input collected at each of the input terminals 110 can be converted to a digital signal. The digital signal can be processed through Fast Fourier transform (FFT) in the DSP 114 to convert the time-domain signal into a frequency-domain signal. In some embodiments, the ECU 116 utilizes the frequency-domain signal calculated by the DSP 114 to determine whether a wire is attached at the input terminal 110. As shown in FIGS. 4A-5B, there is a significant increase in the spectral content associated with the frequency-domain signal when a cable or wire is attached at the input terminal 110A, as compared to the FFT spectrum if no cable or wire is attached at the input terminal 110A. The difference in spectral content is due to the electromagnetic noise that is picked up by the cable or wire when the cable or wire is attached at the input terminal 110A, increasing the frequency content as compared to if no cable or wire is attached at the input terminal 110A.

The spectrum output from the DSP 114 for each channel or each input terminal 110 can be communicated to the ECU 116. The ECU 116 can include, among other things, a cable detection module 122 and a safety module 124. In some embodiments, the cable detection module 122 and/or safety module 124 are implemented by hardware (e.g., application specific integrated circuit (ASIC)) or a combination of hardware/software (e.g., processor executing instructions stored on a memory). The cable detection module 122 can receive the spectrum output from the DSP 114 and use that output to determine whether a cable or wire is attached at one or more of input terminals 110A, 110B and 110C.

The cable detection module 122 can provide an output of “cable detected” or “cable not detected” for each input terminal 110A, 110B and 110C. Specifically, the cable detection module 122 can look at the measured amplitude at a particular frequency or a particular frequency range to determine whether a cable or wire is detected. In some embodiments, the output can be based on a threshold amplitude and the output is “cable detected” if the measured amplitude is greater than the predetermined threshold. In some embodiments, the output is “cable detected” if the cumulative amplitude is above a predetermined threshold over a frequency range.

In some embodiments, the output of “cable detected” or “cable not detected” can be communicated to the display module 106 as a visual notification and/or an audio notification. For example, the display 106 (see FIG. 1) could include an audio alarm if a cable is detected. In another example, the display 106 could include a red visual signal on the display panel if a cable is detected.

In some embodiments, the output from the cable detection module 122 can be provided to the safety module 124, which can be configured to allow or prohibit operation of other components of the system 100, such as the signal generator 118. For example, the safety module 124 can prohibit the signal generator 118 from initiating a start up test (and thereby delivering power to the front panel 108) if a cable is detected. However, once the start-up test has been completed, the system 100 may be ready to deliver IRE pulses from the generator 118 to provide, for example, ablation therapy. Under such scenario, the safety module 124 can then allow operation of the signal generator 118 when the cable is detected in the input terminal 110A.

FIG. 4A is a frequency plot illustrating spectral content for a first channel (see input 110A of FIG. 3) when a wire 120 is attached to the input terminal 110A. FIG. 4B is a similar frequency plot for the first channel when the wire 120 is not attached to the input terminal 110A. As shown in FIG. 4A, measurable noise (i.e. amplitude) from the wire 120 is picked up at frequencies between 0 and 1000 Hertz, particularly between 0 and about 600 Hertz. By contrast, FIG. 4B shows that there is minimum amplitude measured between 0 and 1000 Hertz when the wire 120 is not attached. Thus, a comparison of FIGS. 4A and 4B facilitates detection of the attached wire 120 based on spectral content derived from the FFT signal.

FIG. 5A is a similar frequency plot for channels adjacent to the first channel (see input 110B and 110C of FIG. 3) when the wire 120 is attached to the input terminal 110A. FIG. 5B is a frequency plot for adjacent channels when the wire 120 is not attached to the input terminal 110A. Although the difference in measured noise between FIGS. 5A and 5B is not as significant as the difference between FIGS. 4A and 4B, there is still a notable difference in measured amplitude between FIGS. 5A and 5B given that there is interaction between adjacent channels. When a wire is attached at the first input terminal 110A, adjacent channels may detect a connection, albeit at a lower level. This illustrates that the spectral content of adjacent channels can also be observed in addition to looking at spectral content for the first channel 110A.

Adjacent channels can thus be used to confirm the spectral content of the first channel 110A—if a wire is detected at the input terminal 110A based on the spectral content for the first channel, then adjacent channels should also have some increase in spectral content. In addition, adjacent channels can also be used to determine if there is an issue with the wire 120 at the input terminal 110A such that a connection is not clear from the spectral content for the first channel. For example, if there was a fault for the wire 120 at the input terminal 110A, the spectral content at the first channel 110A could be much lower. In that case, the spectral content of adjacent channels can be evaluated to help determine if a wire is in fact connected at the input terminal 110A. Similarly, if the wire 120 is connected to the input terminal 110A but the latter stages of the first channel are broken, there may be no spectral output on the first channel even though the wire 120 is connected. In other words, there is an interaction between the channel 110A and the channels 110B and 110C such that, if a wire is attached at the channel 110A, it will interact with channels 110B and 110C such that those channels may also have some increase in spectral content.

In the examples described above, each input terminal in FIG. 3 corresponds to a single wire. In that instance, each input terminal corresponds to a single channel. In other examples, an input terminal on a panel of the electrical system can be configured to receive multiple wires. Each wire can have a corresponding receptable in the input terminal and thus the input terminal has multiple receptable to receive the multiple wires. Because each wire corresponds to a channel, in such examples, multiple channels could correspond with a single input terminal.

Cable and wire are used interchangeably herein for describing detection (i.e. wire detection, cable detection) at an input terminal. In some embodiments, a wire can refer to a single wire attached to a device, whereas a cable can refer to a housing for multiple wires attached to the device, and the multiple wires are contained within the cable. As an example, a catheter containing multiple electrodes at an end of the catheter may have a cable connected at the opposite end of the catheter, and the cable may include multiple wires with each wire corresponding to a particular electrode.

FIG. 6 is a flowchart illustrating steps in a method 600 utilized to detect whether a wire or cable is attached at an input terminal of an electrical system. In some embodiments, the electrical system is utilized in conjunction with medical devices such as catheters. The external electrical system may be utilized to collect and analyze data collected from the medical device and/or deliver therapy to the medical device such as ablation therapy. At step 602, the method 600 includes monitoring a signal associated with the input terminal on an input panel of the electrical system. The monitored signal can be an analog signal. In some embodiments, the analog signal is converted to a digital signal using an analog-to-digital converter (ADC) to provide digital signal for downstream processing. At step 604, the digital signal is converted to a frequency-domain signal using a DSP and FFT. At step 606, the frequency domain signal is used to determine or detect whether an external wire or cable is connected to the input terminal. The frequency domain signal includes a spectral output which can be analyzed to determine whether a wire or cable is connected. Such analysis can be based, for example, on the measured amplitude at a specific frequency or over a range of frequencies. In some embodiments, digital signal processing and FFT analysis are already used in operating the electrical system and thus the method 600 is able to use existing hardware/software components and capabilities to determine whether a wire or cable is connected.

At step 608, notification protocols can be generated to relay the results in step 606 as to whether a cable is detected or not detected. Notification protocols can include an audio notification and/or visual notification. Step 608 can also include implementing safety protocols based on the results in step 606. In some embodiments, the safety protocols can include prohibiting operation of other components of the overall system so long as a cable or wire is detected. In some embodiments, the safety protocols can include initiating operation of other components of the overall system if a cable or wire is detected.

For a device having multiple wires attached to it via a cable, the methods and systems disclosed herein can be used to detect whether the various wires are operating properly. For example, a catheter configured for use with a signal generator can contain multiple electrodes and each electrode can have a corresponding wire. The corresponding input terminal on the signal generator or on the electrical system can have multiple receptacles, each receptacle corresponding to a particular wire attached to the catheter. The spectral content for each wire can be analyzed to determine if the wire is connected properly and/or if its corresponding electrode is operating properly. Since each wire (and its corresponding electrode) is associated with a channel in the electrical system, signals collected for each channel can be processed as described above to produce spectral content. The spectral content for each channel can be analyzed to determine whether each wire is connected properly and/or whether its corresponding electrode is operating properly. For example, if power is provided to the catheter and a particular amplitude is not observed at a particular frequency for all channels, it could indicate a wire on the catheter cable is not sufficiently connected at the input terminal or the electrode itself is not operating properly.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

### Discussion of Possible Embodiments

The following are non-exclusive descriptions of possible embodiments of the present invention.

According to one aspect, a method of detecting whether an external wire is connected to an input terminal on an external medical system includes monitoring a signal associated with the input terminal, the input terminal located on an exterior panel of the external medical system and configured for receiving an external wire, and analyzing spectral content of the monitored signal. The method further includes detecting whether the external wire is connected to the input terminal based on the spectral content of the monitored signal.

The method of the preceding paragraph can optionally include, additionally and/or alternatively any, one or more of the following features, steps, configurations and/or additional components.

In some embodiments, the signal associated with the input terminal is a time-domain signal, and the method can further include converting the time-domain signal to a frequency-domain signal using a fast Fourier transform (FFT).

In some embodiments, detecting whether the external wire is connected to the input terminal is based on spectral content derived from the FFT.

In some embodiments, the external wire is determined to be connected if a measured amplitude of the frequency-domain signal is above a predetermined amplitude threshold.

In some embodiments, the external wire is determined to not be connected if a measured amplitude of the frequency-domain signal is less than a predetermined amplitude threshold.

In some embodiments, the external wire is housed within a cable containing additional wires.

In some embodiments, the external wire is connected to a device configured to receive electrical power from the external medical system.

In some embodiments, the external wire is included as part of a catheter.

According to another aspect, a method of operating an electrical system configured to provide power to one or more external components includes monitoring a signal associated with an input terminal on an amplifier system of the electrical system, wherein the input terminal is configured for connection to an external wire, analyzing spectral content of the monitored signal, and determining whether the external wire is connected to the input terminal based on the spectral content of the monitored signal. The method further includes performing at least one of: generating a notification relaying whether the external wire is connected and implementing safety protocols, if the external wire is connected.

The method of the preceding paragraph can optionally include, additionally and/or alternatively any, one or more of the following features, steps, configurations and/or additional components.

In some embodiments, the method further comprises prior to analyzing spectral content, converting the monitored signal to a digital signal and processing the digital signal through FFT.

In some embodiments, the external wire is determined to be connected if a measured amplitude is above a predetermined amplitude threshold.

In some embodiments, the external wire is determined to not be connected if a measured amplitude is less than a predetermined amplitude threshold.

In some embodiments, implementing safety protocols comprises prohibiting electrical power from being delivered from a signal generator of the electrical system.

In some embodiments, generating a notification that the external wire is connected comprises providing at least one of a visual output and an audio output.

According to another aspect, a system configured to provide electrical power to one or more external components includes one or more input terminals formed on an exterior panel of the system, the one or more input terminals configured to receive an external wire, and a data collection system configured to monitor and collect analog signals associated with the one or more input terminals, wherein the analog signals undergo digital signal processing in the data collection system to provide spectral content for the one or more input terminals. The system further includes a cable detection module configured to detect whether the external wire is connected to the one or more input terminals based on the spectral content.

The system of the preceding paragraph can optionally include, additionally and/or alternatively any, one or more of the following features, steps, configurations and/or additional components.

In some embodiments, the system further comprises a safety module configured to allow or prohibit power delivery to the one or more external components, based on whether the cable detection module detects the external wire,

In some embodiments, the one or more external components include a catheter attachable to the one or more input terminals, and the system further comprises a signal generator configured to apply a drive signal to one or more electrodes on the catheter.

In some embodiments, the safety module initiates a self-test of the signal generator if the external wire is not detected.

In some embodiments, the catheter is attachable to the one or more input terminals via a cable, the cable containing multiple wires and each wire corresponds to an electrode on the catheter.

In some embodiments, the cable detection module provides an output of whether the external wire is detected. the output including at least one of a visual output and an audio output.

1. A method of detecting whether an external wire is connected to an input terminal on an external medical system, the method comprising:
monitoring a signal associated with the input terminal, the input terminal located on an exterior panel of the external medical system and configured for receiving an external wire;
analyzing spectral content of the monitored signal; and
detecting whether the external wire is connected to the input terminal based on the spectral content of the monitored signal.

2. The method of claim 1, wherein the signal associated with the input terminal is a time-domain signal, and the method further comprises:
converting the time-domain signal to a frequency-domain signal using a fast Fourier transform (FFT).

3. The method of claim 2, wherein detecting whether the external wire is connected to the input terminal is based on spectral content derived from the FFT.

4. The method of claim 3, wherein the external wire is determined to be connected if a measured amplitude of the frequency-domain signal is above a predetermined amplitude threshold.

5. The method of claim 3, wherein the external wire is determined to not be connected if a measured amplitude of the frequency-domain signal is less than a predetermined amplitude threshold.

6. The method of claim 1, wherein the external wire is housed within a cable containing additional wires.

7. The method of claim 1 wherein the external wire is connected to a device configured to receive electrical power from the external medical system.

8. The method of claim 1, wherein the external wire is included as part of a catheter.

9. A method of operating an electrical system configured to provide power to one or more external components, the method comprising:
monitoring a signal associated with an input terminal on an amplifier system of the electrical system, wherein the input terminal is configured for connection to an external wire;
analyzing spectral content of the monitored signal;
determining whether the external wire is connected to the input terminal based on the spectral content of the monitored signal; and
performing at least one of:
generating a notification relaying whether the external wire is connected; and
implementing safety protocols, if the external wire is connected.

10. The method of claim 9, further comprising: prior to analyzing spectral content, converting the monitored signal to a digital signal and processing the digital signal through FFT.

11. The method of claim 9, wherein the external wire is determined to be connected if a measured amplitude is above a predetermined amplitude threshold.

12. The method of claim 9, wherein the external wire is determined to not be connected if a measured amplitude is less than a predetermined amplitude threshold.

13. The method of claim 9, wherein implementing safety protocols comprises prohibiting electrical power from being delivered from a signal generator of the electrical system.

14. The method of claim 9, wherein generating a notification that the external wire is connected comprises providing at least one of a visual output and an audio output.

15. A system configured to provide electrical power to one or more external components, the system comprising:
one or more input terminals formed on an exterior panel of the system, the one or more input terminals configured to receive an external wire;
a data collection system configured to monitor and collect analog signals associated with the one or more input terminals, wherein the analog signals undergo digital signal processing in the data collection system to provide spectral content for the one or more input terminals; and
a cable detection module configured to detect whether the external wire is connected to the one or more input terminals based on the spectral content.

16. The system of claim 15, further comprising a safety module configured to allow or prohibit power delivery to the one or more external components, based on whether the cable detection module detects the external wire.

17. The system of claim 16, wherein the one or more external components include a catheter attachable to the one or more input terminals, and the system further comprises a signal generator configured to apply a drive signal to one or more electrodes on the catheter.

18. The system of claim 17, wherein the safety module initiates a self-test of the signal generator if the external wire is not detected.

19. The system of claim 17, wherein the catheter is attachable to the one or more input terminals via a cable, the cable containing multiple wires and each wire corresponds to an electrode on the catheter.

20. The system of claim 15, wherein the cable detection module provides an output of whether the external wire is detected, the output including at least one of a visual output and an audio output.
